(19) United States
(12) Patent Application Publication
(10) Pub. No.: US 2008/0144005 A1
(43) Pub. Date: Jun. 19, 2008

(54) METHOD FOR ANALYZING BLOOD CONTENT OF CYTOLOGICAL SPECIMENS

(75) Inventors: Patrick Guiney, Concord, MA (US); Howard Kaufman, Newton, MA (US)

Correspondence Address: VISTA IP LAW GROUP LLP, 12930 Saratoga Avenue, Suite D-2, Saratoga, CA, 95070, US

(73) Assignee: Cytyc Corporation, Marlborough, MA (US)

(21) Appl. No.: 11/958,898

(22) Filed: Dec. 18, 2007

Related U.S. Application Data
(60) Provisional application No. 60/870,840, filed on Dec. 19, 2006.
(60) Provisional application No. 60/870,841, filed on Dec. 19, 2006.

(51) Int. Cl. G01N 33/48

(52) U.S. Cl. 356/39

(57) ABSTRACT

Methods for processing a cytological specimen suspended in a liquid. Light at a wavelength less than 450 nm is directed through the liquid. The intensity of the light transmitted through the liquid is detected and compared to a threshold to determine whether the blood content of the specimen should be reduced before a specimen slide is prepared. In one embodiment, Light at different wavelengths is directed through the liquid. One of the wavelengths is at or near a hemoglobin absorption peak that is less than 450 nm. The respective intensities of light at the different wavelengths that are transmitted through the liquid are detected, and a ratio of the detected intensities is calculated and compared to a threshold to determine whether the blood content of the specimen should be reduced before a specimen slide is prepared

### RELATED APPLICATION DATA

The present application claims the benefit under 35 U.S.C. §119 to U.S. provisional patent applications Ser. Nos. 60/870,840, filed Dec. 19, 2006, and 60/870,841, filed Dec. 19, 2006. The foregoing applications are hereby incorporated by reference into the present application in their entirety for all purposes.

### FIELD OF THE INVENTION

The present invention relates to preparing specimens of biological specimens, and more particularly, to identifying biological specimens having excessive blood content and that should be treated before a specimen slide is prepared.

### BACKGROUND

Medical professionals and technicians often prepare a biological specimen on a specimen carrier, such as a slide, and review the specimen to analyze whether a patient has or may have a particular medical condition or disease. For example, a specimen is examined to detect malignant or pre-malignant cells as part of a Papanicolaou (Pap) smear test and other cancer detection tests. After a specimen slide has been prepared, automated systems are used to focus the technician's attention on the most pertinent cells or groups of cells, while discarding less relevant cells from further review. One known automated slide preparation system that has been effectively used is the ThinPrep processing system available from Cytyc Corporation 250 Campus Drive, Marlborough, Mass. 01752. The test using this system is generally referred to as a ThinPrep (TP) Papanicolaou (Pap) test, or more generally, a ThinPrep or TP test.

Referring to FIG. 1, one known ThinPrep processing system includes a container or vial 10 that holds a cytological specimen 12, a filter 20, a valve 30 and a vacuum source 40. The specimen 12 typically includes multiple cells 14 that are dispersed within a liquid, solution or transport medium 16, such as PreserveCyt, also available from Cytyc Corporation. One end of the filter 20 is inserted into the liquid 16, and the other end of the filter 16 is coupled through the valve 30 to the vacuum source 40. When the valve 30 is opened, vacuum or negative pressure 42 from the vacuum source 40 is applied to the filter 20 which, in turn, draws liquid 16 up into the filter 20. Cells 14 in the drawn liquid 16 are collected by filter 20, as shown in FIG. 2. Referring to FIG. 3, the filter 20 having collected cells 14 is brought into contact with a slide 50. Referring to FIG. 4, the filter 20 is removed from the slide 50, thereby resulting in a specimen slide 50 having a layer of cells 14.

At times, the filter 20 may become clogged when preparing slides 50 of specimens 12 having excessive blood (e.g., lysed blood cells). Clogging of the filter 20 may result in a premature indication that the filter 20 has collected a sufficient number of cells 14 and has sufficient cell 14 coverage. Consequently, the layer of cells 14 that is collected by the filter 20 and applied to the slide 50 may not have the desired number of cells 14 or cell 12 distribution, thereby resulting in an unsatisfactory specimen slide 50.

To address filter clogging, specimen samples 12 with too much blood can be treated with glacial acetic acid to eliminate blood and reduce or prevent clogging of the filter 20. However, selecting and treating specimen samples 12 in an efficient manner can be difficult, time consuming and expensive.

One known technique for selecting specimens for treatment is visually inspecting each vial and making a subjective judgment whether the particular specimen has too much blood and should be treated to reduce blood content. Thus, this technique is essentially based on how much blood is visible in the specimen.

One visual inspection method is described in “A Simple Method to Determine the Need for Glacial Acetic Acid Treatment of Bloody Thinprep Pap Tests Before Slide Processing,” Diagnostic Ctyopathology, Vol. 31, No. 5 (2004), by Leslie R. Rowe, et al. (“the Rowe article”). The Rowe article describes a study that involves visually inspecting each vial and assigning a number to the vial to indicate the amount of blood that is visible in the specimen. A designation of “0” indicates the absence of visible blood, a designation of “1+” indicates that the sample was slightly pink or orange and slightly cloudy, a designation of “2+” indicates that the sample was dark pink to orange and very cloudy, and a designation of “3+” indicates that the sample was dark red and opaque. The conclusion of this study was that specimen samples assigned a designation of 1+ or greater were suitable for a glacial acetic acid wash, and unsatisfactory samples assigned a value of 1+, 2+ or 3+ benefited from processing using glacial active acid.

While visual inspection methods, such as the method described by Rowe, may be useful to a limited extent, these methods are time consuming, require a person to inspect each vial, are based on human judgment, are prone to error and are effective only on a small scale. Further, visual inspection techniques are not automated and typically are not easily integrated within known automated slide processing systems.

Another known system is described in U.S. Pat. No. 4,305,659. This patent describes using two different light sources and an absorbance ratio of fluid based on light transmitted through the fluid at two different wavelengths. However, the described system and method are not suitable for determining whether to treat a specimen 12 to reduce blood content prior to preparing a specimen slide 50 since the patent is directed to detecting the presence of hemoglobin (even small traces). Further, various quantities of blood, including low level traces and some quantities of blood, may be acceptable in a specimen for preparing an acceptable specimen slide. U.S. Pat. No. 4,305,659 also requires alternately energizing illumination sources, adjusting resulting intensities and normalizing and determining the ratio of transmission intensities, and these controls.

### SUMMARY

One embodiment of the invention is directed to a method of processing a cytological specimen suspended in a liquid to determine whether the blood content of the specimen should be reduced before a slide containing the specimen is prepared. The method according to this embodiment includes directing light at a first wavelength through the cytological specimen and directing light at a second wavelength through the cytological specimen. The first wavelength is less than 450 nm, and the second wavelength is longer than the first wavelength. The intensities of light at the first and second wavelengths transmitted through the cytological specimen are detected, and a ratio of the first and second intensities is calculated and compared to a pre-determined threshold. Based on the comparison, a determination is made whether the blood content should be reduced before a slide containing cells of the cytological specimen is prepared.

According to another embodiment of the invention, a method of processing a cytological specimen suspended in a liquid to determine whether the specimen should be treated before a slide containing the specimen is prepared includes directing light at a first wavelength through the cytological specimen and directing light at a second wavelength through the cytological specimen. The first wavelength is about 405 nm, and the second wavelength is longer than the first wavelength. The method further includes detecting the intensities of light at the first and second wavelengths transmitted through the cytological specimen and calculating a ratio of the first and second intensities. The ratio is compared to a pre-determined threshold, and based on the comparison, a determination is made whether the blood content should be reduced before a slide containing cells of the cytological specimen is prepared. If the blood content should be reduced, the cytological specimen is treated. A specimen slide can then be prepared, and a filter is inserted into the liquid which is held in a container, and a vacuum is applied to the filter to collect cells of the treated cytological specimen. Cells of the treated specimen are collected and applied to the slide. Otherwise, if the blood content is acceptable, processing can proceed by inserting a filter into the liquid held in the container, applying a vacuum to the filter to collect cells of the untreated cytological specimen, and applying collected cells of the untreated cytological specimen to the slide.

In accordance with a further embodiment of the invention, a method of processing a cytological specimen suspended in a liquid to determine whether the blood content of the specimen should be reduced includes directing light at a first wavelength of about 405 nm from a first light emitting diode through the cytological specimen and directing light at a second wavelength from a second light emitting diode through the cytological specimen. The second wavelength is longer than the first wavelength. The method also includes detecting the intensity of light at the first wavelength transmitted through the cytological specimen and detecting the intensity of light at the second wavelength transmitted through the cytological specimen. The first and second intensities are advantageously detected without a spectrophotometer. A ratio of the first and second intensities is calculated and compared to a pre-determined threshold. Based on the comparison, a determination is made whether the blood content of the specimen should be reduced. If so, the cytological specimen is treated with glacial acetic acid. Then, a filter is inserted into the liquid of the treated specimen, a vacuum is applied to the filter to collect cells of the treated cytological specimen, and collected cells of the treated specimen are applied to the slide. Otherwise, if it is determine that the blood content is acceptable, a filter is inserted into the liquid which is held in a container, a vacuum is applied to the filter to collect cells of the untreated cytological specimen, and collected cells of the untreated cytological specimen are applied to the slide.

In various embodiments, hemoglobin absorbs a substantial amount of light at the first wavelength, e.g., the first wavelength is at or near a hemoglobin absorption peak that is less than 450 nm, whereas the second wavelength is longer than the first wavelength and may or may not be at or near a hemoglobin absorption peak. If the second wavelength is at or near another hemoglobin absorption peak, the absorption of hemoglobin at the first, shorter wavelength being substantially greater than absorption of hemoglobin at the second, longer wavelength.

In various embodiments, the first wavelength is about 405 nm±15 nm. The second wavelength is at least 30% longer than the first wavelength and can be about 525 nm or about 630 nm. The light sources can be, for example, light emitting diodes or white light with filters at different wavelengths. If necessary, the optical path length through a container or vial holding the cytological specimen can be reduced to accommodate the particular light source(s) used. Embodiments can be performed without the use of a spectrophotometer and other additional processing steps used in known devices, such as alternating between the first and second wavelengths varying an intensity of the detected light at the first or second wavelengths.

According to yet another embodiment of the invention, a method of processing a cytological specimen suspended in a liquid to determine whether the specimen has excessive blood includes directing light at a wavelength less than 450 nm through the cytological specimen and detecting the intensity of light transmitted through the cytological specimen. The intensity is compared to a pre-determined threshold, and based on the comparison, a determination is made whether the blood content of the cytological specimen should be reduced before a slide containing cells of the cytological specimen is prepared.

In still another embodiment of the invention, a method of processing a cytological specimen suspended in a liquid to determine whether a sample has excessive blood content includes directing light at a wavelength of about 405 nm through the cytological specimen and detecting the intensity of light transmitted through the cytological specimen. The method also includes comparing the intensity to a pre-determined threshold and, based on the comparison, determining whether the blood content of the cytological specimen should be reduced before a slide containing cells of the cytological specimen is prepared. If so, the cytological specimen is treated to reduce blood content. A filter is inserted into the liquid, which is in a the container, and a vacuum is applied to the filter to collect cells of the treated cytological specimen. Collected cells of the treated cytological specimen are applied to the slide. Otherwise, if the blood content is acceptable, a filter is inserted into the liquid held by the container, and a vacuum is applied to the filter to collect cells of the untreated cytological specimen. Collected cells are applied to the slide.

In a still a further embodiment of the invention, a method of processing a cytological specimen suspended in a liquid to determine whether the blood content of the specimen should be reduced includes directing light at a wavelength of about 405 nm from a light emitting diode through the cytological specimen and detecting the intensity of light that is transmitted through the specimen. Detection is performed without a spectrophotometer. The intensity is compared to a pre-determined threshold and, based on the comparison, a determination is made whether the cytological specimen should be treated to reduce blood content before a slide containing cells of the cytological specimen is prepared. If so, the cytological specimen is treated with, for example, glacial acetic acid to reduce blood content in the cytological specimen, and a filter is inserted into the liquid which is held in a container for processing. A vacuum is applied to the filter to collect cells of the treated cytological specimen, and collected cells are applied to the slide.

In yet another embodiment of the invention, a method of processing a cytological specimen suspended in a liquid held in a container includes directing light at a wavelength less than 450 nm through a cytological specimen having blood and cells and directing the light through liquid that does not include blood. A first intensity of the light transmitted through the cytological specimen having blood and cells is detected, and a second intensity of the light transmitted through the liquid that does not include blood is detected. The first and second intensities are compared to determine whether the blood content of the cytological specimen should be reduced before a slide containing cells of the cytological specimen is prepared.

In various embodiments, the light source is a light emitting diode, and hemoglobin absorbs a substantial amount of light at the first wavelength less than 450 nm, which can be at or near a hemoglobin absorption peak that is less than 450 nm. In one embodiment, the first wavelength is about 405 nm±15 nm. If necessary, the container or vial can have a reduced optical path length to accommodate various slight sources.

In various embodiments, blood content determinations are made before filtering the cytological specimen, and if the blood content is too high, then the specimen can be treated, e.g., with glacial acetic acid. Further, if necessary, containers have specimens containing excessive blood can be separated from other specimens.

Further, in certain embodiments in which intensities of light passed through liquid containing blood and cells and liquid that does not contain blood, the cytological specimen having blood and cells can be contained in a first vial, and the liquid having no blood is contained in another vial. Alternatively, specimens can be mixed and also allowed to settle to allow light to be directed through a specimen having blood and cells as well as to allow light to be directed through liquid that does not include blood since the blood and other solids will settle to the bottom of the container when the specimen is not mixed and is allowed to settle.

### BRIEF DESCRIPTION OF THE DRAWINGS

Referring now to the drawings in which like reference numbers represent corresponding parts throughout, and in which:

FIG. 1 illustrates a known slide preparation system and method that use a cytological filter for collecting cells and applying a layer of collected cells to a specimen slide;

FIG. 2 is a bottom view of a known cytological filter with collected cells to be applied to a specimen slide;

FIG. 3 illustrates a known method of applying cells collected by a cytological filter to a specimen slide;

FIG. 4 shows a specimen slide having a layer of cells applied by a cytological filter;

FIG. 5 illustrates a system for processing a specimen according to one embodiment;

FIG. 6 further illustrates a system for processing a specimen according to one embodiment;

FIG. 7 illustrates a system having two light sources and one detector according to one embodiment;

FIG. 8 illustrates a system having two light sources and two detectors according to one embodiment;

FIG. 9 is a flow chart of a method for processing a specimen using two light sources according to one embodiment;

FIG. 10 is a flow chart of a method for processing a specimen after determining whether a specimen should be treated according to one embodiment;

FIG. 11 is a chart illustrating the known absorption spectra of hemoglobin;

FIG. 12 is another chart illustrating known absorption spectra of hemoglobin;

FIG. 13 illustrates a test system that uses three different light sources and a detector and demonstrates effectiveness of embodiments of the invention;

FIG. 14 shows intensity data of transmitted light acquired using the system shown in FIG. 13;

FIG. 15 is a graph of the data shown in FIG. 14;

FIG. 16 is a graph of the data shown in FIG. 14 in the form of a ratio of measurements involving a vial containing blood to an average intensity of blank vials having no blood;

FIG. 17 is a graph of the data shown in FIG. 16 in logarithmic form;

FIG. 18 is a graph of data showing an absorption by hemoglobin in specimen samples having different concentrations of blood;

FIG. 19 is a graph of data showing an absorption by hemoglobin in specimen samples having different concentrations of blood that appeared bloody based on visual inspection;

FIG. 20 is a graph of data showing an absorption by hemoglobin in specimen samples having different concentrations of blood and a specimen preservative having no blood;

FIG. 21 is a graph of data showing an absorption by hemoglobin in specimen preservative with a plastic strip in the optical path having blood and having no blood;

FIG. 22 illustrates a modified internal structure of a vial to provide a reduced optical path length according to one embodiment;

FIG. 23 illustrates a system having one light source and one detector according to one embodiment;

FIG. 24 is a flow chart illustrating a method of processing a specimen using the system shown in FIG. 23 according to one embodiment;

FIG. 25 is a system diagram illustrating light transmitted through specimen samples having blood and cells and a reference liquid according to one embodiment;

FIG. 26 is a flow chart illustrating a method of processing a specimen using the system shown in FIG. 25 according to one embodiment;

FIG. 27 is a system diagram illustrating light transmitted through a vial containing a mixture of liquid, blood and cells and light transmitted through only liquid in the vial after cells and blood have settled to the bottom of the vial according to one embodiment; and

FIG. 28 is a flow chart illustrating a method of processing a specimen using the system shown in FIG. 27 according to one embodiment.

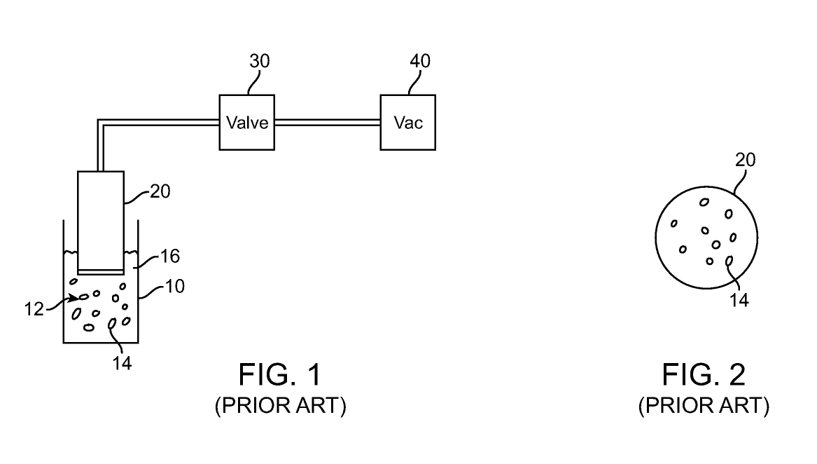

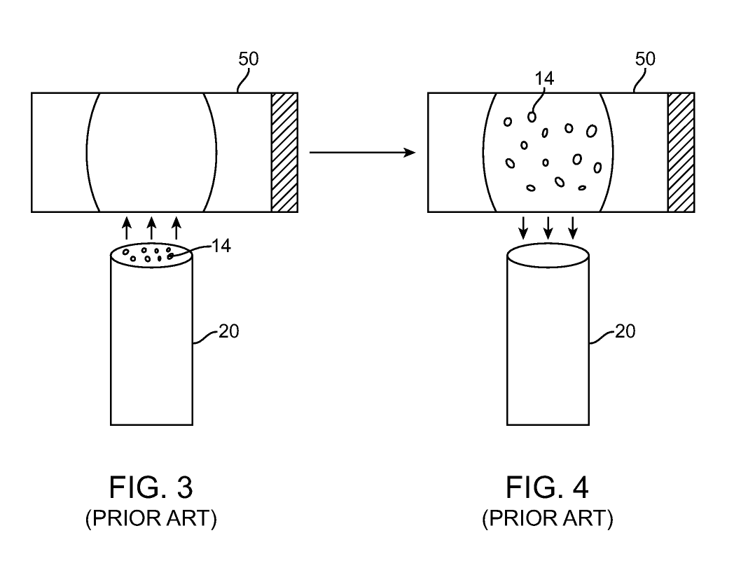

### DETAILED DESCRIPTION OF ILLUSTRATED EMBODIMENTS

In the following description, reference is made to the accompanying drawings which form a part hereof, and which show by way of illustration specific embodiments and how they may be practiced. It is to be understood that changes may be made without departing from the scope of embodiments.

Embodiments are directed to systems and methods for determining which biological specimens have too much blood and should be treated to reduce blood content before a specimen slide is prepared. Embodiments improve known systems and methods by providing one or more light or illumination sources (generally light sources) that are arranged to direct light through the specimen. If two light sources are used, the light sources are at different wavelengths.

At least one wavelength is at or near an absorption peak of hemoglobin, or about 415 nm±about 15 nm and a second wavelength is a longer wavelength, e.g., at least 30% longer than 415 nm. A detector measures the intensities of different wavelengths of light that pass through the specimen. A controller calculates a ratio of intensities and compares the ratio to a threshold, which represents an acceptable amount of blood in the specimen. The comparison of the ratio to the threshold indicates whether the specimen should be treated to reduce blood content. Thus, with embodiments of the invention, it is not necessary to calculate the amount of hemoglobin or blood present in a specimen sample and it is not necessary to analyze spectral curves since embodiments compare intensities of light and whether a threshold has been exceeded.

In embodiments involving one light source, it is not necessary to calculate a ratio of intensities. Rather, the detector measures the intensity of light that passes through the specimen, and a controller compares the measured intensity to a threshold, which represents an acceptable amount of blood in the specimen. The wavelength of light used in the single light source embodiment is at or near an absorption peak of hemoglobin, or about 415 nm±about 15 nm.

In the following description, reference is made to the accompanying drawings, which show by way of illustration how specific embodiments may be practiced. It is to be understood that other embodiments may be utilized as various changes may be made without departing from the scope of embodiments. In this specification, references to “first” and “second” components, such as first and second light or illumination source sources, emitted light, transmitted light, wavelengths and detectors are intended to refer to different light sources, different emitted light, different transmitted light, different wavelengths and different detectors. Accordingly, the terms “first” and “second” are not intended to refer to any particular order of method steps or particular magnitudes. Thus, for example, a first light source is not necessarily activated first, and a first wavelength is not necessarily shorter than other wavelengths.

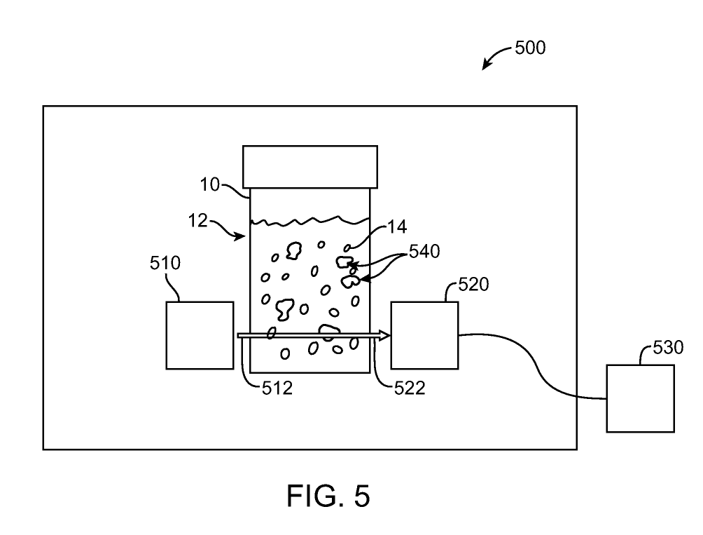

Referring to FIG. 5, a system 500 according to one embodiment includes a one or more light sources 510 and one or more detectors 520. FIG. 5 generally illustrates one light source 510 and one detector 520. It will be appreciated upon reading this specification that the system 500 can include one light source 510 that emits light at one wavelength or multiple light sources 510 that emit light 512 at different wavelengths. Further, the system 500 can include one detector 520 that detects light 522 transmitted through the vial 10 at one wavelength or one detector 520 that detects light 522 transmitted through the vial 10 at multiple wavelengths or multiple detectors that detect transmitted light at multiple wavelengths 520, e.g., a separate detector 520 for each light source 510.

A container 10, such as a vial, holds the specimen 12 and is located between the one or more light sources 510 and the one or more detectors 520. Light 512 emitted from the one or more light sources 510 is directed into the vial 10 and the specimen 12. Light 522 transmitted through the vial 10 and the specimen 12 and is detected by one or more detectors 520, which measure the intensity of the transmitted light 522. The intensity measurements are provided to a controller 530. The controller 530 can be, for example, analog circuitry, a processor, a computer, a micro-controller, or a logic device, such as a programmable logic device.

The controller 530 processes the intensity data to determine whether specimen 12 contains too much blood 540 (e.g., lysed blood cells) and, therefore, should be treated to reduce blood content 540. For purposes of illustration, blood 540 is shown as being larger than specimen cells 12.

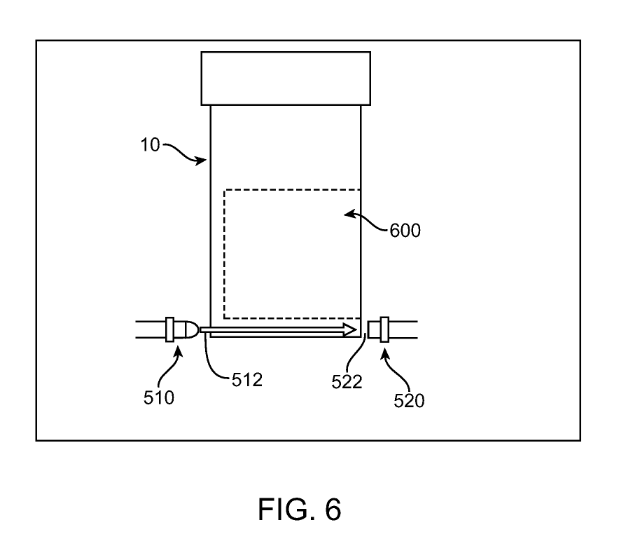

Referring to FIG. 6, according to one embodiment, the one or more light sources 510 include one or more Light Emitting Diodes (LED's), and the one or more detectors 520 include one or more photodetectors. Preferably, the detector 520 is not a spectrophotometer since a spectrophotometer can be expensive and a relatively large instrument that requires periodic calibration and particular algorithms. According to another embodiment the light source can be a broadband white light source, such as a xenon or tungsten lamp, with one or more wavelength specific notch filters.

As shown in FIGS. 5 and 6, the vial 10 may or may not include a label 600 attached thereto. A light source 510 can be arranged to emit light 512 through various portions of the vial 10 if the vial does not include a label. As shown in FIG. 6, if the vial 10 includes a label 600, then a light source 510 can be arranged to emit light 512 below or around the label 600. Alternatively, if the label 600 only wraps partially around the vial 10, e.g., less than 50% around the vial 10, then the vial 10 and a light source 510 can be arranged so that the light source 510 emits light 512 through various uncovered sections of the vial 10. Accordingly, the arrangement shown in FIG. 6 is provided for purposes of explanation and illustration, not limitation.

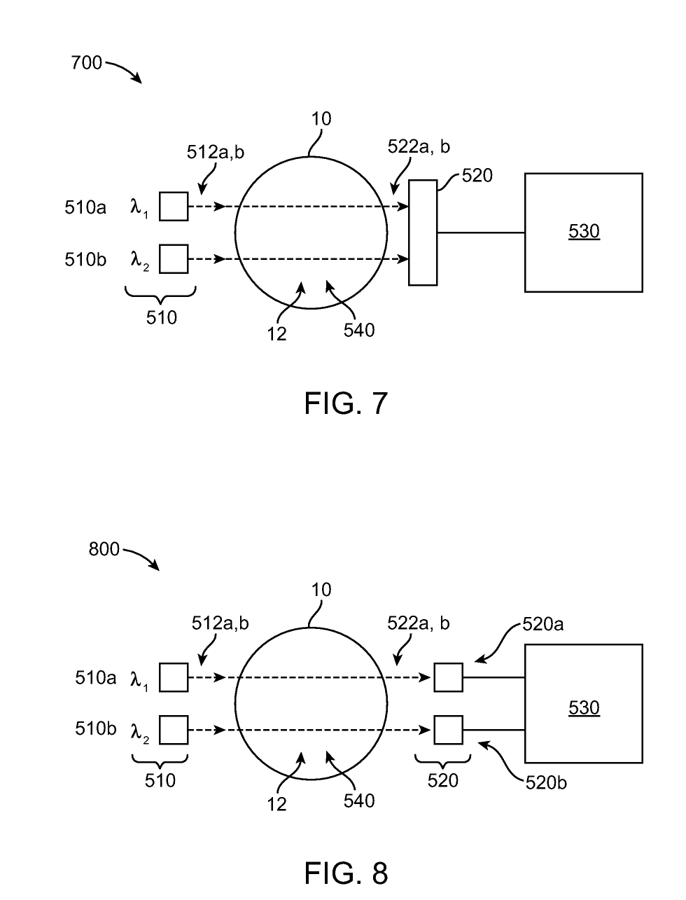

Referring to FIG. 7, according to one embodiment, a system 700 for analyzing the blood content of a specimen to determine whether the specimen should be treated prior to preparing a specimen slide includes two light sources 510a and 510b (generally light source(s) 510) and one detector 520. A first light source 510a emits light 512a at a first wavelength λ1, and a second light source 510b emits light 512b at a second wavelength λ2. The detector 520 detects light 522a at the first wavelength λ1 that passes through the vial 10 and the specimen 12 and detects light 522b at the second wavelength λ2 that passes through the vial 10 and the specimen 12. In this embodiment, the same detector 520 is used to detect the intensity of transmitted light 522 at the first and second wavelengths λ1 and λ2.

Referring to FIG. 8, according to another embodiment, a system 800 for analyzing the blood content of a specimen to determine whether the specimen should be treated prior to preparing a specimen slide includes two light sources 510a and 510b and two detectors 520a and 520b. A first light source 510a emits light 512a at a first wavelength λ1, and a second light source 510b emits light 512b at a second wavelength λ2. A first detector 520a detects light 522a at the first wavelength λ1 that passes through the vial 10 and the specimen 12, and a second detector 520b detects light 522b at the second wavelength λ2 that passes through the vial 10 and the specimen 12. The first and second light sources 510 and the first and second detectors 520 are arranged so that light 512 emitted from the light sources 510 at different wavelengths is detected by the detectors 520 positioned opposite the light sources 510.

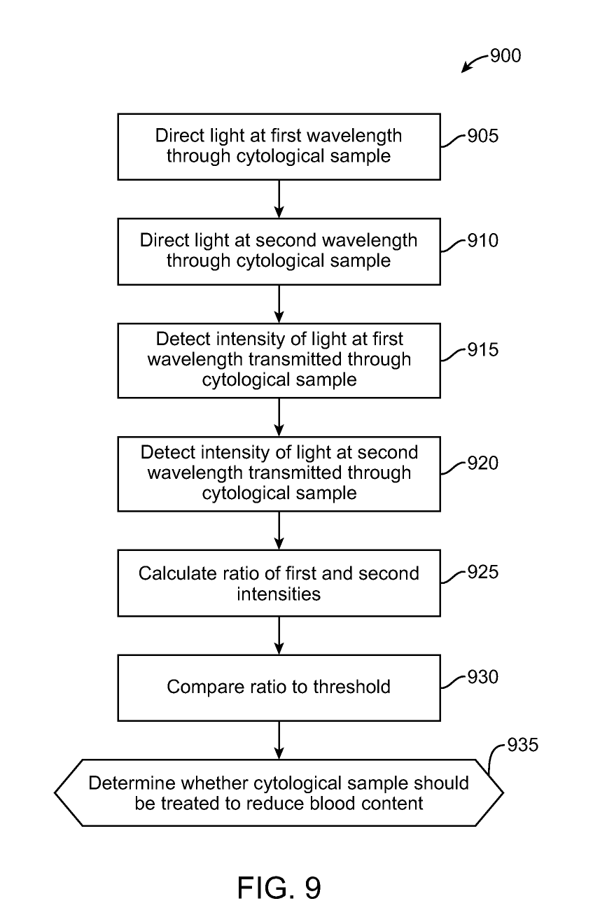

FIG. 9 illustrates a method 900 for analyzing a biological specimen for determining whether the specimen should be treated prior to preparing a specimen slide according to one embodiment. The method can be implemented using a system having two light sources, such as the systems 700 and 800 shown in FIGS. 7 and 8. In step 905, light at a first wavelength is directed from a first light source and through the vial and specimen. In step 910, light at a second or reference wavelength is directed from a second light source and through the vial and specimen. The second wavelength is different than the first wavelength. For example, the first wavelength can be the shortest wavelength, and the second wavelength can be longer than the first wavelength.

In step 915, light at the first wavelength that is transmitted through the specimen is detected by the detector, which measures the intensity of the light at the first wavelength. In step 920, light at the second wavelength that is transmitted through the specimen is detected by the detector, which measures the intensity of the light at the second wavelength. In step 925, a controller calculates a ratio of the intensities of light at the first and second wavelengths. According to one embodiment, the ratio is (intensity of light at first wavelength)/(intensity of light at second wavelength). Alternatively, the ratio may calculated by dividing the intensity of light at the second wavelength by the intensity of light at the first wavelength. In step 930, the calculated ratio is compared to a pre-determined threshold, and in step 935, a determination is made whether the specimen should be treated to reduce the blood content in the specimen based on the comparison of the ratio and the threshold.

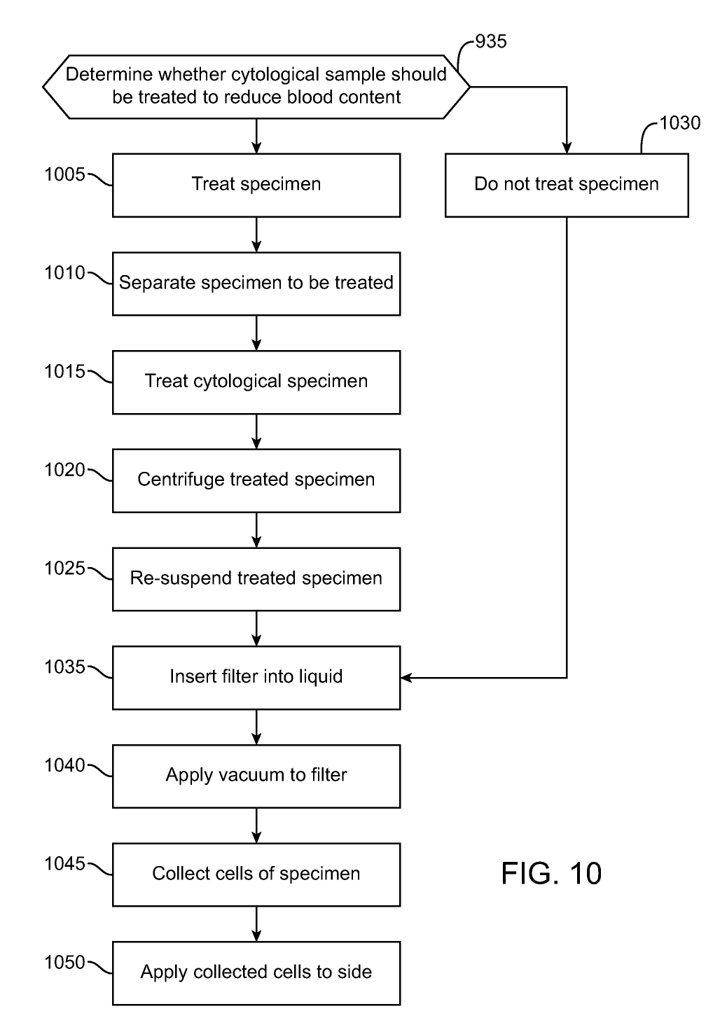

Further steps following the determination step 935 are shown in FIG. 10. Referring to FIG. 10, if it is determined 1005 that the blood content of the specimen is too high and should be reduced by treating the specimen, then in step 1010, if necessary, the specimen that is to be treated can be separated from other specimens that do not require treatment. Thus, one option is to separate or triage specimens to be treated from other specimens so that the other specimens can be processed without further delay. Alternatively, step 1010 may not be required if treatment can be performed in-line. In step 1015, the selected specimen is treated to reduce the blood content in the specimen. According to one embodiment, this is performed using glacial acetic acid, e.g. by adding glacial acetic acid to the specimen. In step 1020, if necessary, the treated sample can be centrifuged. In step 1025, if necessary, the treated sample can be re-suspended in the liquid to ensure a desired distribution and suspension of cells in the liquid.

After steps 1005-1025, the specimens can be processed in the same manner as other specimens that were not treated or triaged. Thus, whether it is determined in step 1005 that the specimen should be treated or it is determined in step 1030 that no treatment is necessary, continuing with step 1035, a filter is inserted into the liquid in which the treated (or untreated) specimen is suspended. In step 1040, vacuum is applied to the filter to draw liquid and cells of the treated (or untreated) specimen up through the filter. In step 1045, cells of the treated (or untreated) specimen are collected by the filter, and in step 1050, the collected cells of the treated (or untreated) specimen are applied to a cytological specimen carrier, such as a slide, to prepare a specimen slide having a layer of cells. Thus, embodiments advantageously identify selected specimens having excessive blood, treat the selected specimens to reduce blood content and prepare acceptable specimen slides.

According to one embodiment, a first wavelength of light 512 emitted by a first light source 510 is at or near an absorption peak of hemoglobin. In one embodiment, the first wavelength is less than 450 nm, e.g., about 415 nm. The first wavelength can vary from 415 nm while remaining near the absorption peak of hemoglobin, e.g., about 415 nm±about 15 nm. Light at these wavelengths is generally referred to as “violet” light. The second wavelength is longer than the first wavelength. According to one embodiment, the second wavelength is at least 30% longer than the first wavelength. According to one embodiment, the second wavelength is at another, less prominent hemoglobin absorption peak. According to one embodiment, the second wavelength is at about 530 nm to about 580 nm. Light at these wavelengths is generally referred to as “green” light. Alternatively, the second wavelength can be at a wavelength which is at a still weaker hemoglobin absorption peak. The second wavelength can be at a wavelength that is weakly absorbed by hemoglobin, e.g., at a wavelength of about 630 nm to about 680 nm. Light at these wavelengths is generally referred to as “red” light. Other wavelengths can also be used for purposes of calculating a ratio. Further, white light can be used. For example, a ratio of the intensity of “violet” light to the intensity of “white” light can be utilized in an alternative embodiment.

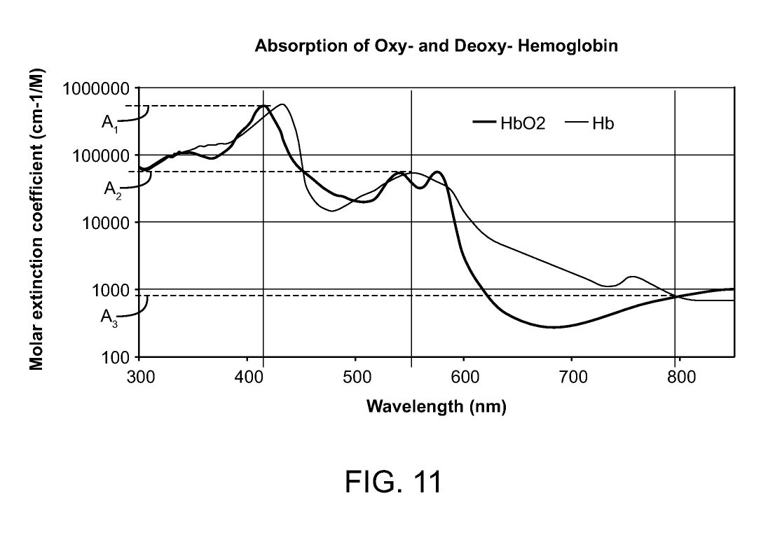

FIGS. 11-21 illustrates tests that were performed that validate embodiments of the invention and that demonstrate the advantages that are achieved using violet light at a wavelength of about 415 nm. While these tests used a spectrophotometer to generate spectral absorption curves for analysis, with embodiments of the invention, it is not necessary to calculate the amount of hemoglobin or blood present in a specimen sample. Further, it is not necessary to generate or analyze spectral curves.

Instead, embodiments compare intensities of light of predetermined wavelengths and whether a threshold has been exceeded, and the spectral curve analysis was performed to demonstrate the effectiveness of using violet light at 415 nm and to demonstrate how different blood concentrations can be analyzed to determine a threshold value. Once a threshold value is determined, it is not necessary to consult spectral curves since subsequent use involves a transmitted intensity or comparing a ratio to the threshold value.

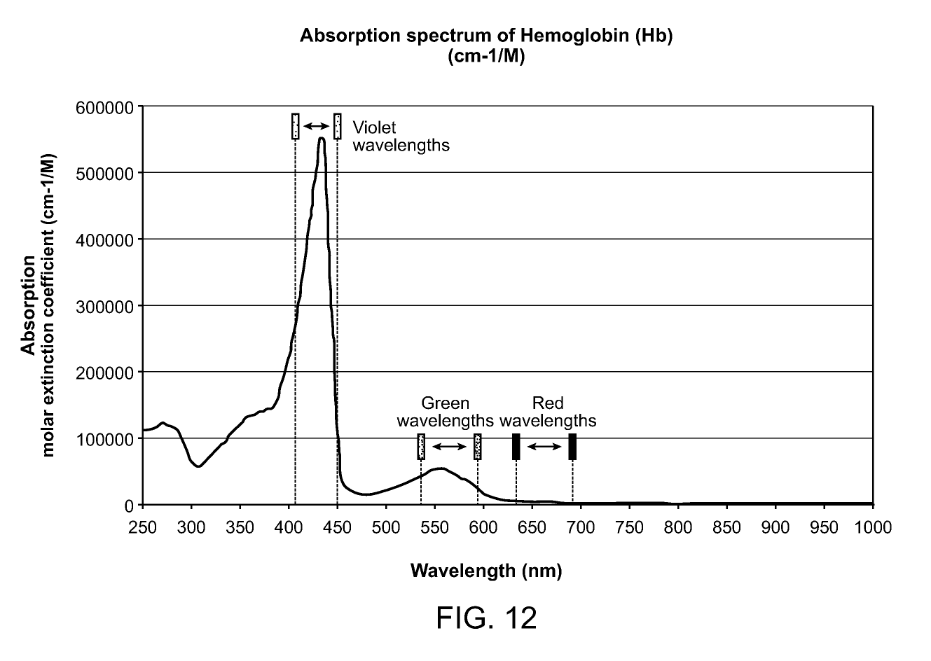

FIGS. 11 and 12 illustrate the hemoglobin absorption peaks that are utilized by embodiments of the invention. FIG. 11, as published by the Oregon Medical Laser Center, is one known chart that shows the known absorption spectra for oxygenated and deoxygenated hemoglobin as a function of wavelength. In FIG. 11, the y-axis scale is logarithmic, and the absorption A1, A2 and A3 by oxygenated and deoxygenated hemoglobin vary at different wavelengths of about 415 nm (“violet” light) and about 550 nm (“green” light). More specifically, the absorption A1 is substantially higher at a wavelength of about 415 nm±15 nm compared to the absorption at A2 at a wavelength corresponding to a weaker absorption peak. As shown in FIG. 11, the absorption decreases significantly at wavelengths higher than about 630 nm (“red” light). More specifically, the molar extinction coefficient is a maximum value at A1 at a wavelength of about 415 nm. The next highest molar extinction coefficient is at an intermediate value of about 550 nm. In other words, hemoglobin absorbs substantially more “violet” light than “green” light. Hemoglobin absorbs considerably less “red” light than “violet” light as shown by the graph at wavelengths between 650 nm and 800 nm.

FIG. 12 further illustrates the absorption spectra of hemoglobin with different y-axis values to illustrate in further detail how the magnitude of absorption of light at by hemoglobin various at different wavelengths and how much more “violet” light is absorbed by hemoglobin compared to “green” and “red” light. As shown in FIG. 12, the molar extinction coefficient of hemoglobin is about 55,000 cm−1/M, whereas the molar extension coefficient of hemoglobin is about 5,000 cm−1/M. Thus, as shown in FIG. 12, hemoglobin absorbs more than 10 times as much “violet” light at about 415 nm as it does at “green” light at about 550 nm. Embodiments of the invention advantageously utilize these absorption characteristics to identify specimens having excessive blood and that should be treated prior to preparing a specimen slide.

With reference to the spectra shown in FIGS. 11 and 12, a ratio of the intensity of transmitted light at a first wavelength to the intensity of light at a second or reference wavelength can be, for example, a ratio of the intensity of transmitted light at about 415 nm±15 nm (violet) to the intensity of transmitted light at about 550 nm±30 nm (green). In an alternative embodiment, the ratio is a ratio of the intensity of transmitted light at 415 nm±15 nm (violet) to the intensity of transmitted light at 650 nm±30 nm (red). In a further alternative embodiment, the ratio is a ratio of the intensity of transmitted light at about 415 nm±15 nm to transmitted white light.

Once a ratio is calculated using light at about 415 nm±15 nm relative to another wavelength or type of light, the ratio is compared to a threshold. Threshold values can be determined according to various criteria, with the result that slides exceeding the threshold should be treated to reduce excessive blood content. For example, threshold values can be determined by testing different specimen samples having different concentrations of blood. Ratios of the intensities of light transmitted at different wavelengths through these samples are calculated, and the ratio of the specimen sample having the most blood that can still result in an acceptable slide is assigned to be the threshold ratio or threshold value. The intensity of light at 415 nm (violet) can be measured, and the intensity of light at a second, reference wavelength, such as 525 nm (green) can be measured at the determined threshold. Thus, subsequent ratio calculations involving violet and green light can be compared to the threshold to determine which samples have excessive blood and should be treated to reduce blood content, e.g., with a glacial acetic acid wash. Ratio and threshold determinations can be based on various combinations or ratios of light at various wavelengths.

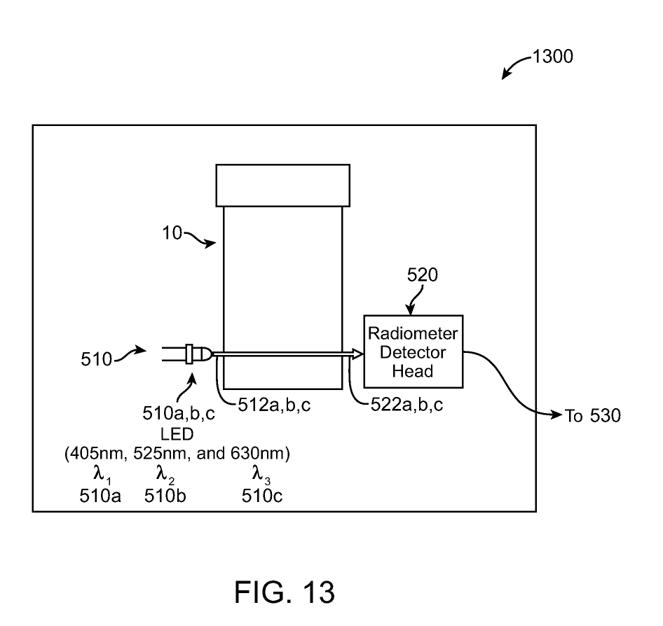

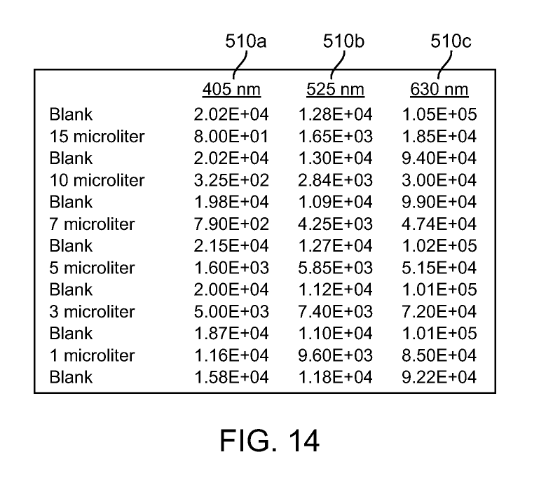

FIG. 13 illustrates a test system 1300 that was used to demonstrate effectiveness of and validate embodiments of the invention by showing how a threshold value can be determined and how a ratio and threshold can be compared. The test system 1300 used three different light sources 510, i.e., three LED's 510a, 510b and 510c, and a single detector 520, i.e., a broadband detector or radiometer. Other detectors other than a radiometer can also be utilized, including a photodiode. One suitable photodiode is Part No. VTB8440, available from PerkinElmer, 45 William Street, Wellesley, Mass. 02481 USA.

Output light from each LED 510 was centered at a different wavelength. Output light from a first LED 510a was centered at a 405 nm, output light from a second LED 510b was centered at 525 nm and output light from a third LED was 510c was centered at 630 nm. The 405 nm LED 510a was Part No. L200CUV405-8D, available from Ledtronics, Inc., 23105 Kashiwa Ct., Torrance, Calif. 90505. The 525 nm LED 510b was Part No. ETG-5MN525-15, available from ETG Corporation, 8599 Venice Boulevard, Unit K, Los Angeles, Calif. 90034. The 630 nm LED 510c was Part No. ETG-5TS630-15, also available from ETG Corporation in Los Angeles, Calif. The radiometer 520 that was used was model no. IL-1700, and the detection head for the radiometer 520 was Model No. SED-033, both of which are available from International Light Technologies, 10 Technology Drive, Peabody, Mass. 01960. The calibration factor for the radiometer 520 was 1.594×e−10. A 405 nm “notch” optical filter (not shown) with a bandwidth of approximately 10 nm was also used when the “violet” LED 510a was utilized. The optical filter was model no. 405FS10-12.5, and is available from Andover Corporation, 4 Commercial Drive, Salem, N.H. 03079.

Specimen samples having different concentrations of blood 540 were prepared by adding different quantities of blood to six vials containing the same amount of liquid or solution 16. Each vial included 20 ml of PreserveCyt solution and a different quantity of whole blood—1 microliter, 3 microliters, 5 microliters, 7 microliters, 10 microliters and 15 microliters. The specimen samples were swirled for about 10 seconds prior to taking any measurements.

Each of the six vials 10, one vial 10 at a time, was positioned between a single LED 510 and the detector 520. The intensity of the light transmitted through the vial was measured. A first set of measurements was made with the violet LED (405 nm) 510a, a 4.0 volt bias and a 28 mA drive current. Optical measurements were conducted with the 405 nm optical notch filter in the detector head (with and without room lights) and without the filter in place (with and without the room lights). A second set of measurements was made with the green LED (525 nm) 510b, a 3.5 volt bias and a 20 mA drive current. A third set of measurements was made with the red LED (630 nm) 510c, a 2.1 volt bias and a 27 mA drive current. Additionally, before and after each measurement of a blood-containing vial 10, a measurement was also made using a “blank” vial containing only 20 ml of PreserveCyt solution (no blood and no label). Thus, three measurements were acquired for each of the six vials 10.

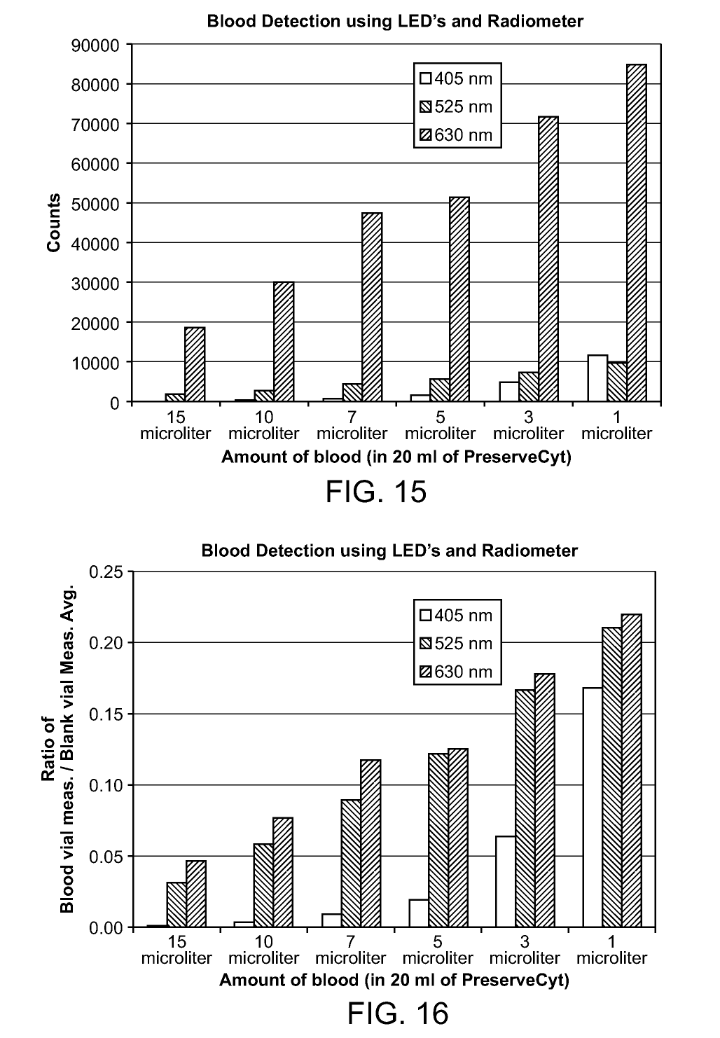

FIG. 14 shows the data acquired during this test at different wavelengths and for different blood concentrations. FIG. 15 shows this data in chart form in terms of the number of counts or how much light passed through the vials 10 having different quantities of blood and detected by the detector 520 for each tested wavelength.

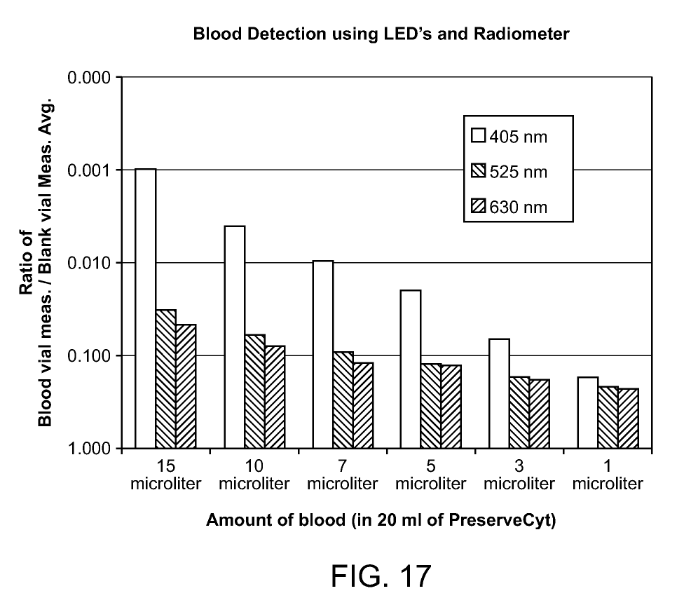

During these tests, a small amount of drift in the power output of the LEDs 510 was observed. To minimize the effects of the power output drifts, a ratio was calculated of the blood vial measurement over the average of the preceding and subsequent “blank” vial measurements having no blood—[Blood vial meas./(Blank VialPrec.+Blank VialSubs./2)]. This data is plotted in FIG. 16. FIG. 17 illustrates the data in FIG. 16 in logarithmic form.

The data and charts shown FIGS. 14-17 illustrate that greater quantities of light passed through the solution/blood mixture when a vial included less blood. The data and charts also show that the amount of 415 nm light that passed through the samples was generally substantially lower than the amount of light at other wavelengths given the high absorption of 415 nm light by hemoglobin compared to other wavelengths.

A threshold can be selected based on the resulting specimen slide that is prepared for a sample having a given concentration of blood. Thus, if an acceptable specimen slide was prepared from a specimen having five microliters of blood, but an unacceptable slide was prepared from a specimen having seven microliters of blood, then a threshold can be set based on the five microliters specimen. Thus, for example, the intensity of light at 415 nm (violet) can be measured, and the intensity of light at a second, reference wavelength, such as 525 nm (green) can be measured. The ratio of the intensity at 415 nm to the intensity of light at 525 nm can establish a threshold value. Thus, subsequent ratio calculations involving violet and green light can be compared to the threshold to determine which samples have excessive blood and should be treated, e.g., with a glacial acetic acid wash. Persons skilled in the art will appreciate that the ratio and threshold determinations can be based on various combinations or ratios of light at various wavelengths.

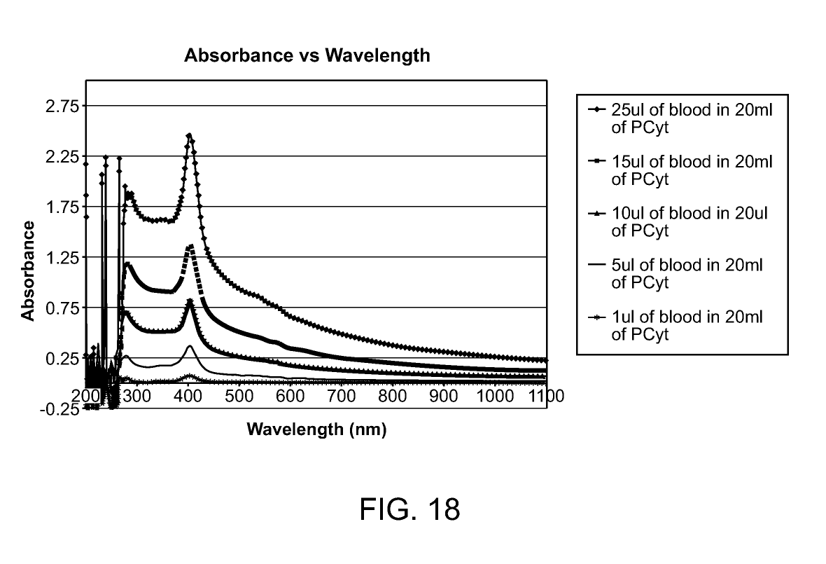

FIGS. 18-21 show the results of another test that validates embodiments of the invention and show how a threshold value may be determined against which a ratio is compared. This test was conducted using a Shimadzu 1601UV spectrophotometer. Cell-free samples of PreserveCyt solution 16 in combination with different quantities of blood 540 and cells 14 were prepared and pipetted into a glass spectrophotometer cuvette with an optical pathlength of 10 mm. The following samples cell-free PreserveCyt solution and whole blood were prepared: 1 μl of blood in 20 ml of PreservCyt; 5 μl of blood in 20 ml of PreservCyt 10 μl of blood in 20 ml of PreservCyt; 15 μl of blood in 20 ml of PreservCyt and 25 μl of blood in 20 ml of PreservCyt. All samples were vortexed before measurements were taken.

In addition to these five samples, a sample vial was selected from approximately 300 vials from which unsatisfactory slides were produced. The sample vial was selected based on its “bloody” appearance (by visual inspection). A series of dilutions was prepared from the chosen sample. After each spectral measurement was taken, approximately half of the volume of the glass cuvette was removed and replaced with fresh PreservCyt. This led to the following approximate concentrations: undiluted original sample; diluted 1:2 (1 part original in 2 parts of final solution); diluted 1:4 (1 part original in 4 parts of final solution); diluted 1:8 (1 part original in 8 parts of final solution) and diluted 1:1 (1 part original in 16 parts of final solution). All samples were mixed by repeated pipetting before measurements were taken.

A sample from a “normal” appearing vial (i.e. with cells but with no apparent blood, as observed by eye) and a sample of fresh PreservCyt were selected to act as “control” samples. A cuvette of fresh PreservCyt with a strip of vial material (approximately 7 mm wide by 50 mm long) inserted in the cuvette with and without blood mixed with the PreserveCyt.

FIG. 18 is a graph showing data that was collected and shows the absorbance (logarithmic function of optical transmission) as a function of wavelength for each of the five different samples having different concentrations of blood in PreserveCyt solution. FIG. 18 shows the absorption peak at around 410-415 nm and that the amplitude of the peak at about 410 nm varies depending on the concentration of blood. The absorbance scale of the y-axis is logarithmic so at the highest concentration (the top spectral line representing 25 μl of blood to 20 ml of PreserveCyt) there is a strong absorbance and a very low level of transmittance [Absorbance(λ)=log(100/% T)].

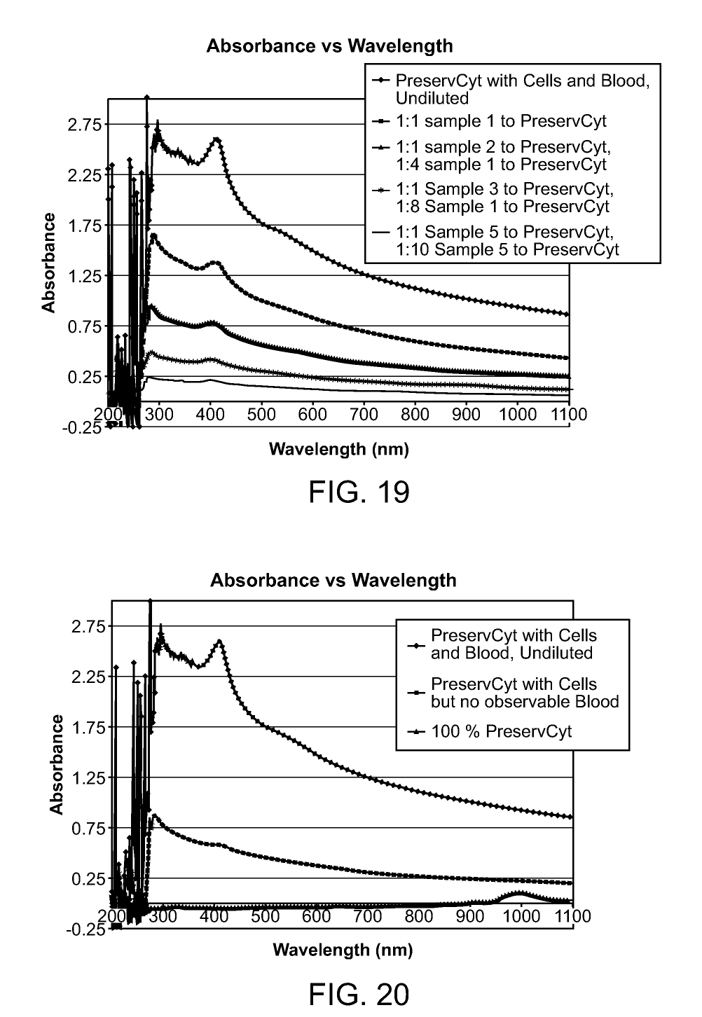

FIG. 19 is a graph showing data collected from one undiluted concentration of a specimen (top spectral line) and four diluted concentrations of a specimen (four bottom spectral lines) that contains visible blood. FIG. 19 illustrates a similar absorption peak at about 410 nm for all of the samples. FIG. 19 also shows curves having a slight offset and peaks that are dampened when the samples are more diluted. For example, the top curve has peak that is more pronounced and sharper than the lower curves. Further, the locations of higher peaks are shifted slightly to the right compared to lower peaks. The dampening and peak shifts may be caused by cells, mucous and other substances. Nevertheless, 410 nm (±10 nm) remains a wavelength that can be used to establish a threshold to indicate whether a specimen contains excessive blood.

FIG. 20 contains data collected from a glass cuvette that was filled with 100% fresh PreservCyt (the bottom spectral line), a glass cuvette that was filled with a sample that appears normal, or that has no observable blood (middle spectral line) and, for reference, the top spectral line from FIG. 19, which represents an undiluted sample that was taking from a vial containing a specimen that had visible blood. FIG. 20 provides additional evidence that an absorption offset is a function of cells and other material. Further, the middle spectral line of FIG. 20, representing a sample having no visually observable blood, has a slight peak at about 410 nm. This slight peak indicates possible presence of some blood even though blood was not observable based on visual inspection.

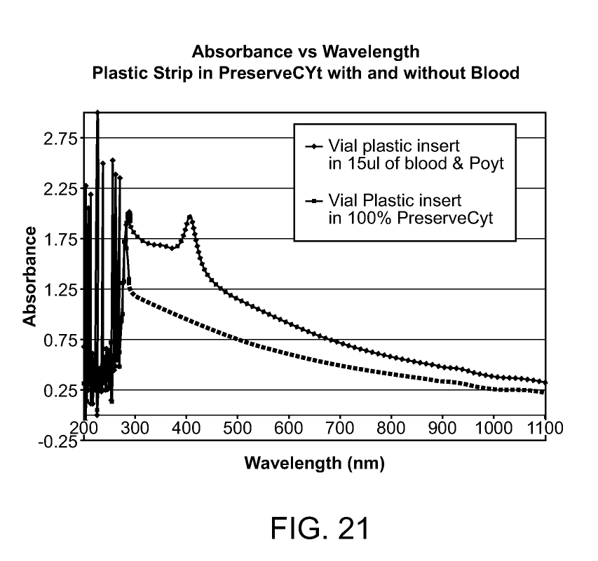

FIG. 21 contains the data collected from a glass cuvette filled with 100% fresh PreservCyt (bottom spectral line) and a strip of vial material (top spectral line) (approximately 7 mm wide by 50 mm long) that was inserted in the cuvette. FIG. 21 demonstrates that optical transmission through the plastic material of the vial is possible for purposes of analyzing blood content of specimens.

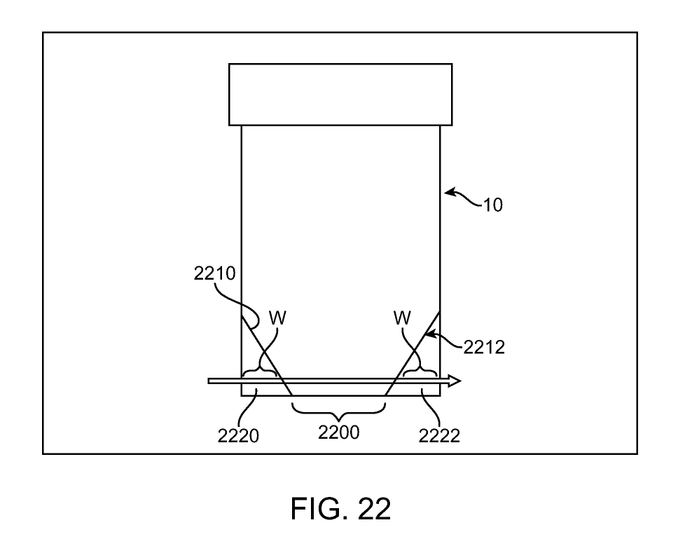

Referring to FIG. 22, according to one embodiment, the internal structure of a vial 10 or container can be modified to accommodate various light sources 510. For example, in some instances, the power of a light source 510 may be less than what is necessary to allow emitted light 512 to be transmitted through the entire vial 10 and the specimen 12. In these instances, the structure of the vial 10 can be modified to provide a section with reduced optical path 2200 length to allow the emitted light 512 from the light to traverse a shorter distance through the vial 10/specimen 12.

According to one embodiment, the reduced optical path length 2200 is achieved by adding two internal walls 2210 and 2212 (generally 2210) that extend between two internal vial surfaces of the vial 10. In the illustrated embodiment, the internal wall 2210 defines a first gap 2220, and the internal wall 2212 defines a second gap 2222. The gaps 2220 and 2222 can, for example, be filled with air or another low absorption substance or medium.

In the illustrated embodiment, each internal wall 2210 extends between a side or vertical wall of the vial 10 and a bottom surface of the vial 10. Thus, the optical path length is reduced by twice the width W of a gap 2220 at a height H, resulting in reduced optical path length 2200.

In the illustrated embodiment, two internal walls 2210 and 2212 are each the same shape and size and define symmetrical gaps 2220 and 2222 that define a reduced optical path length 2200 through the vial 10. In alternative embodiments, different numbers, shapes and arrangements of internal walls 2210 or other internal structures can be utilized to define a reduced optical path length through the vial 10. For example, rather than having two internal walls 2210 and 2212, another embodiment includes one internal block that rests on a bottom surface of the vial 10. The internal block may or may not contact one of the upwardly extending side walls of the vial and, therefore, may define one gap or two gaps, which may or may not be symmetrical. Thus, the configuration shown in FIG. 22 is provided for purposes of explanation and illustration, and is not intended to be limiting since other shapes, numbers and arrangements of internal walls or other structures can be utilized for the purpose of reducing optical path length.

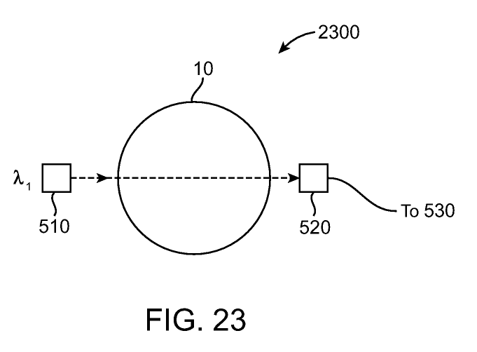

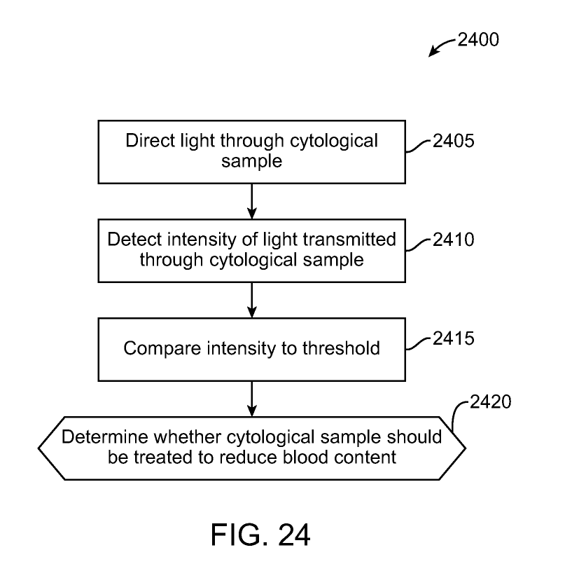

Referring to FIGS. 6 and 23, in a further alternative embodiment, a system 2300 includes a single light source 510 rather than two light sources as shown in FIGS. 7 and 8. Referring to FIG. 24, a method 2400 of processing a biological specimen using a system as shown in FIGS. 6 and 23 includes directing light from the light source and through the vial or specimen in step 2405. In step 2410, light from the light source is transmitted through the vial and is detected by the detector, which measures the intensity of the transmitted light. If necessary, the internal walls of the vial can be modified, as shown in FIG. 22, if the power output of the light source is not sufficient. In step 2415, a controller compares the measured intensity to a pre-determined threshold. In step 2420, the controller determines whether the specimen should be treated to reduce the blood content in the specimen based on the threshold comparison. Thus, in this embodiment, it is not necessary to detect light at two different wavelengths and calculate a ratio of the intensities of light at different wavelengths.

If it is determined that the blood content of the specimen is too high and that the specimen should be treated, then steps 1005 to 1025 and 1035-1050 shown in FIG. 10 can be performed as necessary. Otherwise, if the blood content is acceptable and no treatment is required, then the specimen can be processed as it normally would by performing steps 1030-1050 as shown in FIG. 10.

According to one embodiment, the system 2300 and method 2400 shown in FIGS. 23 and 24 can be implemented using a LED 510. According to one embodiment, the LED 510 emits light 512 at a wavelength that is at or near a peak hemoglobin absorption wavelength. For example, the LED 510 can have a center wavelength of about 405 nm (±15 nm), which is near a maximum absorption peak of hemoglobin as shown in FIG. 12. A single photodetector 520 positioned at the opposite side of the specimen vial 10 measures the amount of violet light 522 that is transmitted through the vial 10.

Embodiments advantageously are capable of using a single light source 510 by taking advantage of the pronounced absorption by hemoglobin of 415 nm light. In other words, because the absorption is so high in the violet bandwidth, a threshold level of transmitted violet light can be used to identify specimens that have excessive quantities of blood that may clog a filter. Accordingly, changes in the intensity of light 522 at a single wavelength that is transmitted through a vial 10 can be observed to indicate the amount of hemoglobin in the specimen which, in turn, indicates the amount of blood in the specimen and whether it is necessary to treat the specimen to reduce blood content before preparing a specimen slide.

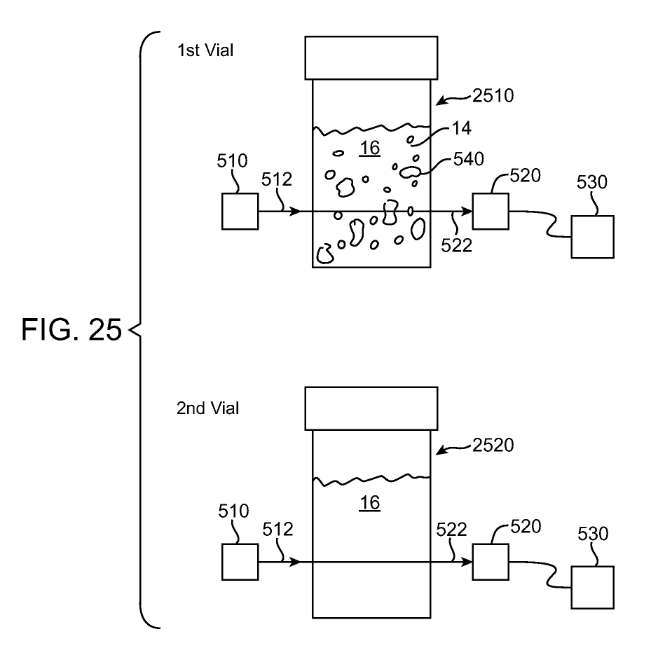

Referring to FIG. 25, in a further alternative embodiment, a system 2500 includes a light source 510 that emits light 512 at a wavelength of about 415 nm. The intensity of the light that is transmitted 522 through a first vial 2510 that contains cells 14 and blood 540 is detected by a detector 520. The same or a different light source 510 emits light 512 that is transmitted through a second or control vial 2510, which serves as a reference. The control vial 2510 contains a liquid 16, such as PreserveCyt solution with cells, but no blood. The intensity of light transmitted through the specimen vial 2510 and the intensity of light transmitted through the control vial 2520 are provided to a controller 530, which compares the intensities to determine whether the blood content in the specimen is too high and should be treated before preparing a specimen slide.

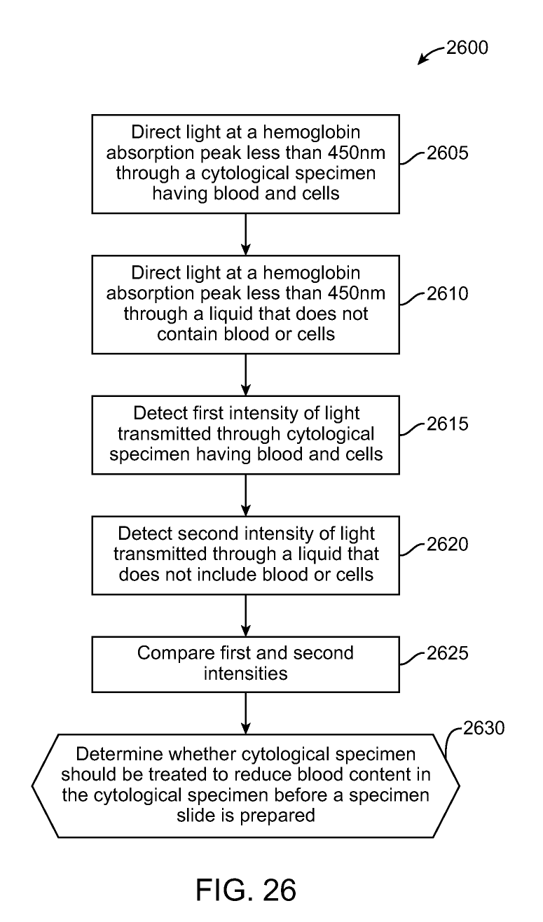

Thus, referring to FIG. 26, a method 2600 for processing a cytological specimen includes, in step 2605, directing light through a cytological specimen having blood and cells. The light is at a wavelength that is at or near a hemoglobin absorption peak less than 450 nm, e.g., 415 nm. In step 2610, light is directed through a liquid that does not include blood. According to one embodiment, a wavelength of light that is directed through the liquid is the same as the wavelength of light directed through the cytological specimen. The light can be emitted by the same light source or different light sources. In step 2615, a first intensity of light that is transmitted through the cytological specimen having blood and cells is detected, and in step 2620, a second intensity of light that is transmitted through the liquid that does not include blood is detected. In step 2620, the first and second intensities are compared, and in step 2625, a determination is made based on the comparison whether the cytological specimen should be treated to reduce blood content in the cytological specimen before a slide containing the cytological specimen is prepared.

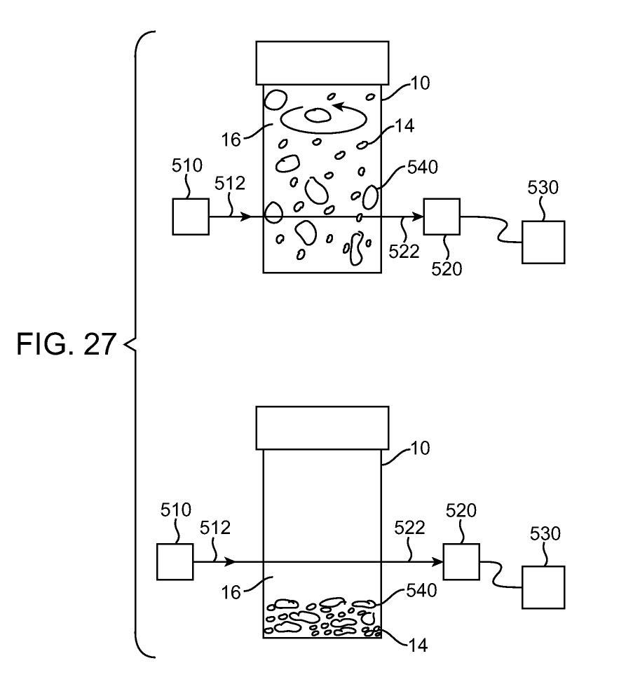

Referring to FIG. 27, rather than using both a specimen vial 2510 and a control vial 2520 (as shown in FIG. 25), in an alternative embodiment, the same vial is used. One measurement is taken when the blood, cells and liquid are mixed together, and another measurement is taken when blood and cells settle to the bottom of the vial so that light is directed only through liquid. More particularly, a system 2700 includes a light source 510 that emits light 512 at a wavelength of about 415 nm. The cells 14, blood 540 and liquid 16 in the vial are mixed together or vortexed (as indicated by arrow) in a second or reference vial 2510 so that the blood 540 and cells 14 are distributed throughout the liquid 16. The vial 2520 includes a liquid 16, such as PreserveCyt solution, but no cells or blood.

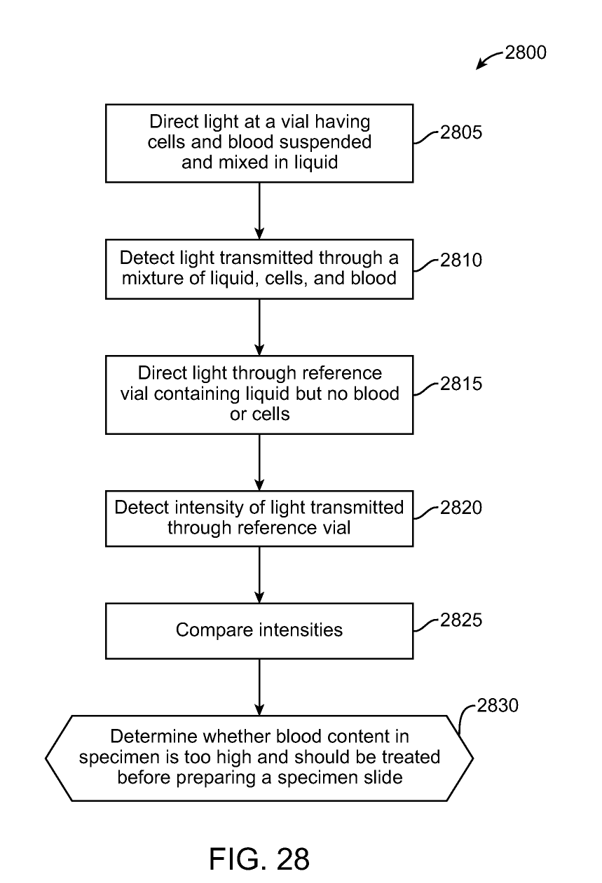

In use, referring to FIG. 28, a method 2800 for processing a cytological specimen according to one embodiment includes in step 2805, directing light through the vial 2710 having cells 14 and blood 540 that are suspended and mixed in a liquid 16. In step 2810, light that is transmitted through the mixture of liquid, cells and blood is detected by a detector. In step 2815, the same or a different light source 510 emits light 512 that is directed through the transmitted through a reference vial 2510, which contains a liquid 16, such as PreserveCyt solution, but no blood. In step 2820, the intensity of light that is transmitted through the reference vial 2510 is detected and serves as a reference intensity against which intensity measurements involving cells and blood are compared. In step 2825, the intensity of light transmitted through the specimen vial 2510 and the intensity of light transmitted through the control vial 2520 are provided to a controller 530, which compares the intensities. In step 2830, the controller determines, based on the comparison, whether the blood content in the specimen is too high and should be treated before preparing a specimen slide.

A further embodiment of the invention is directed to a method for verifying that a clear optical path through the vial exists and detecting the presence of other debris or other non-cellular components in the specimen that could interfere with the filter and collection of cells. From time to time, a sample collection brush is inadvertently left in the specimen vial. This may result in opaque or partially opaque optical path. Other types of debris include lubricant used in the process of obtaining a sample and cervical mucous. Embodiments of the invention that incorporate a reference measurement relative to the transmission of violet light at about 415 nm through the vial to identify debris or other components. A reference measurement to which the actual intensity of transmitted violet light can be compared can be a predetermined level of room light, “white light” or any non-violet light transmitted through the vial. The reference could also be visual or image based.

Embodiments of the invention significantly improve upon known visual inspection systems and methods that determine the blood content of a specimen. Further, embodiments of the invention significantly improve upon known “ratio” systems and methods by eliminating the need for additional steps of alternately energizing illumination at different wavelengths, adjusting the relative intensities of illumination at a first and second wavelength and normalizing and ratioing transmission intensities at two different wavelengths. Instead, embodiments advantageously consider the intensities of the transmitted light, either from a single light source or two light sources, without the need for subsequent manipulation of the intensity data.

Although particular embodiments have been shown and described, it should be understood that the above discussion is not intended to limit the scope of these embodiments. Various changes and modifications may be made without departing from the scope of the claims. Thus, embodiments are intended to cover alternatives, modifications, and equivalents that fall within the scope of the claims.

What is claimed is:

1. A method of processing a cytological specimen suspended in a liquid, comprising:
directing light at a first wavelength through the liquid, the first wavelength being less than 450 nm;
directing light at a second wavelength longer than the first wavelength through the liquid;
detecting an intensity of light at the first wavelength transmitted through the liquid;
detecting an intensity of light at the second wavelength transmitted through the liquid;
calculating a ratio of the detected first and second wavelength light intensities;
comparing the ratio to a pre-determined threshold; and
based on the comparison, determining whether the blood content of the cytological specimen suspended in the liquid should be reduced before preparing a cell sample slide.

2. The method of claim 1, wherein the first wavelength is at a hemoglobin absorption peak below 450 nm.

3. The method of claim 2, wherein the second wavelength is at a hemoglobin absorption peak above 450 nm, absorption of hemoglobin at the first wavelength being substantially greater than absorption of hemoglobin at the second wavelength.

4. The method of claim 2, wherein the second wavelength is not at a hemoglobin absorption peak.

5. The method of claim 1, wherein the first wavelength is on a range of about 390 nm to 420 nm.

6. The method of claim 1, wherein the second wavelength is about 525 nm or about 630 nm.

7. The method of claim 1, wherein the second wavelength is at least 30% greater than the first wavelength.

8. The method of claim 1, wherein directing light at the first wavelength and directing light at the second wavelength comprises:
directing light from a first light emitting diode through the liquid; and
directing light from a second light emitting diode through the liquid.

9. The method of claim 1, wherein the intensity of light at the first and second wavelengths is detected without using a spectrophotometer.

10. The method of claim 1, wherein the calculated ratio comprises a ratio of the first intensity to the second intensity or a ratio of the second intensity to the first intensity.

11. The method of claim 10, further comprising reducing the blood content of the cytological specimen if the calculated ratio is greater than the pre-determined threshold.

12. The method of claim 11, wherein the blood content of the speciment is reduced by treating the specimen with glacial acid.

13. The method of claim 1, wherein a determination of whether the blood content of the cytological specimen should be reduced is performed before filtering the cytological specimen to collect cells for slide preparation.

14. The method of claim 1, performed without alternating between the first and second wavelengths.

15. The method of claim 1, performed without varying an intensity of the detected light at the first or second wavelengths.

16. The method of claim 1, wherein the liquid is held in a container, and wherein light at the first wavelength is directed through a portion of the container having a reduced optical path length relative to other portions of the container.

17. A method of processing a cytological specimen suspended in a liquid held in a container, the method comprising:
directing light having a wavelength less than 450 nm through the container;
detecting the intensity of light transmitted through the container;
comparing the intensity to a pre-determined threshold; and
based on the comparison, determining whether the blood content of the cytological specimen should be reduced before preparing a slide containing cells of the cytological specimen.

18. The method of claim 17, wherein the wavelength is at a hemoglobin absorption peak less than 450 nm.

19. The method of claim 17, wherein the first wavelength is in a range of about 390 nm to 420 nm.

20. The method of claim 17, wherein the light is directed from a light emitting diode through the container.

21. The method of claim 17, wherein the intensity of light is detected without using a spectrophotometer.

22. The method of claim 17, wherein if a determination is made that the blood content of the cytological specimen should be reduced, the method further comprises:
treating the cytological specimen with glacial acetic acid.

23. The method of claim 17, wherein the light is directed through a portion of the container having a reduced optical path length relative to other portions of the container.

24. A method of processing a cytological specimen suspended in a liquid held in a container, comprising:
directing light having a wavelength less than 450 nm through a cytological specimen having blood and cells;
directing the light through liquid that does not include blood;
detecting a first intensity of the light transmitted through the cytological specimen having blood and cells;
detecting a second intensity of the light transmitted through the liquid that does not include blood; and
comparing the first and second intensities to determine whether the blood content of the cytological specimen should be reduced before preparing a slide containing cells of the cytological specimen.

25. The method of claim 24, wherein the cytological specimen and liquid that does not contain blood are held in the same container.

26. The method of claim 24, wherein the wavelength is a hemoglobin absorption peak less than 450 nm.

27. The method of claim 24, wherein the wavelength is in a range of about 390 nm to 420 nm.

28. A method of processing a cytological specimen suspended in a liquid held in a container, the method comprising:
directing light at a first wavelength of about 405 nm from a first light emitting diode through the container;
directing light at a second wavelength from a second light emitting diode through the container, the second wavelength being longer than the first wavelength;
detecting the intensity of light at the first wavelength transmitted through the container;
detecting the intensity of light at the second wavelength transmitted through the container, the first and second intensities being detected without using a spectrophotometer;
calculating a ratio of the first and second intensities;
comparing the ratio to a pre-determined threshold;
determining, based on the comparison, whether the blood content of the cytological specimen should reduced before a slide containing cells of the cytological specimen is prepared based on the comparison; and
if a determination is made that blood content should be reduced before slide preparation,
treating the cytological specimen with glacial acetic acid to reduce the blood content.
